(19) United States
(12) Patent Application Publication
(10) Pub. No.: US 2010/0179599 A1
(43) Pub. Date: Jul. 15, 2010

(54) Device for reducing a bone fracture, in particular a fracture at the bone epiphyses

(76) Inventors: Guillaume Derouet, St. Herblain (FR); Gregoire Larche, Cholet (FR)

Correspondence Address: MCHALE & SLAVIN, P.A., 2855 PGA BLVD, PALM BEACH GARDENS, FL, 33410, US

(21) Appl. No.: 12/352,368

(22) Filed: Jan. 12, 2009

(51) Int. Cl. A61B 17/70; A61B 17/04

(52) U.S. Cl. 606/280; 606/286; 606/301

(57) ABSTRACT

The device according to the invention, for reducing a fracture, in particular a fracture at bone epiphyses, comprises:
an osteosynthesis plate with an elongated body part prolongated by a single-piece head part, said head part including at least some orifices adapted for “poly-axial” assembly of fastening screws,
a targeting guide type device fitted with through-orifices adapted for acting as a guide to said fastening screws,
means for drilling orifices with a set of suitable drill bits,
a first drilling guide suitable to cooperate with one of said orifices of the plate head, to allow the drilling into the bone of a pre-drill-hole by a “poly-axial” type technique, and
a second drilling guide adapted for co-operating with one of the orifices of said targeting guide positioned on said plate head so as to enable the realisation of a pre-drill-hole in the bone by a “mono-axial” type technique.

This application is a continuation in part of application Ser. No. ______, filed on ______, entitled “A Device for Reducing a Fracture in Particular a Distal Radius Fracture”, the contents of which are hereby expressly incorporated by reference.

The present invention relates a device, or more accurately a set of devices (or kit) offered to surgeons for reducing a bone fracture, in particular a fracture at bone epiphyses (distal radius, proximal humerus, distal femur, etc.).

There are numerous systems such as plate(s) associated with a set of fastening screws, offered to surgeons for reducing a bone fracture.

According to the type of fracture at hand, to provide optimal reduction, it may be interesting to tilt the axis of one or of each fastening screw, appropriately, relative to the perpendicular of the supporting plate.

To do so, and for simple fractures, current or relatively current, certain plates comprise “mono-axial” type fastening screws, with a preset orientation. For easy placement of these screws, it is known to use a device called “targeting guide” or “fast drilling guide” (also named in French as “bloc-canon”), added removably on the supporting plate, fitted with a plurality of through-orifices, intended each to come in regard of one of the orifices of the supporting plate and whereof the axis is tilted correctly in relation to the tilting requested for the corresponding screw.

This targeting guide hence enables a predetermined configuration for the associated fastening screws; it is used, in combination with a so called “drilling gun” or “drilling guide”, for acting as a guide for drilling in the material of the screw implanting drill-holes (surgical motor and drill bit), and for acting as a guide to the screws properly speaking when being placed.

For complex fractures, the surgeon may use plates fitted with so-called “poly-axial” fastening screws, i.e. whereof the implantation angle into the bone can be adjusted along an admissible predetermined tilting range.

According to the case at hand, the surgeon may then select the tilting of the fastening screws which it judges as optimal.

These screws are placed after realisation of a drill-hole into the bone, by means of the drilling device, associated with a drilling guide correctly oriented in advance according to the fastening direction selected.

Moreover, in all cases, regardless whether it is a “mono-axial” or “poly-axial” technique type osteosynthesis implant, it is interesting to lock the fastening screw, once fully inserted into the bone, for optimising the mechanical stability of the osteosynthesis assembly thus obtained and limiting the disassembly risks thereof (migration of the screws . . . ) during the bone consolidation phase, after placing the bone under load.

Thus, in practice, according to the fracture that he wishes to reduce, the surgeon makes a choice between the mono-axial or poly-axial type systems available to him, said choice being made generally during the surgical operation, after reduction of the fracture.

The techniques using the “mono-axial” type systems with targeting guide are less costly and quicker to be implemented, however they do not offer surgeons nearly any latitude for implanting the screws.

On the other hand, the “poly-axial” type systems provide the surgeon with a choice for implanting the screws, but they are more costly and require longer implementation time.

The purpose of the present invention is to optimise the surgeon's intervention, in particular for fractures located at bone epiphyses, while offering him a set of devices which he may use regardless of the type of fracture at hand, and this, to suit each fastening screw, according to a poly-axial or mono-axial type technique.

According to the invention, the corresponding device comprises:
an osteosynthesis supporting plate including a bottom face and a top face, said bottom face intended for being located against the receiving bone material; this plate comprises an elongated body part, prolongated by a single-piece head part, wherein said body part comprises a plurality of through-orifices and said head part comprises a plurality of through-orifices, at least some of said through-orifices enabling to accommodate poly-axial type screws, i.e. capable of being implanted along an admissible predetermined tilting range, said plate still comprising, at the linking zone between its body part and its head part, at least one complementary threaded orifice,
a set of screws for fastening into the bone, intended for being inserted into said through-orifices of the plate body, so-called “body screws”, for attaching said plate body at the surface of the bone, which body screws comprise a head prolongated by a body fitted with a thread for fastening in the bone,
a set of screws for fastening into the bone, intended for being inserted into said through-orifices of the plate head, so-called “head screws”, for attaching said plate head at the surface of the bone,
a targeting guide type device intended for being positioned on the top face of said plate head, to enable set tilting positioning of said head screws, which targeting guide consists in a piece or a block of mater having a plurality of through-orifices, identical in number to those of said plate head, each intended for matching one of said orifices of said plate head, which through-orifices are adapted for acting as a guide for said head screws, and which targeting guide still comprises at least one through-orifice intended for being positioned in the extension of said complementary orifice of the plate head, for its removable fixture thereon by means of a suitable fastening screw,
means for drilling orifices, of surgical motor type associated with a set of suitable drill bits,
a first drilling guide, adapted for co-operating with the orifices of said plate head, to enable drilling in the bone a drill-hole for receiving said head screws, according a poly-axial technique, and
a second drilling guide, adapted for co-operating with the orifices of said targeting guide positioned on said plate head, to enable drilling in the bone a drill-hole for receiving one of said head screws, according a mono-axial technique.

Preferably the device according to the invention comprises an osteosynthesis plate whereof at least one of the through-orifices of the head part is fitted with means provided for locking the associated head screw on said plate, upon completion of the screwing operation into the receiving bone material.

According to an advantageous embodiment, at least one of the through-orifices of the head part of the osteosynthesis plate comprises a housing provided for accommodating and holding a nut, wherein said nut is blocked into rotation in its housing relative to said supporting plate and said nut comprises a spherical contact surface capable of co-operating with a complementary spherical contact surface provided in its reception housing, to confer said nut a degree of freedom in said housing, along an admissible predetermined tilting range, to enable tilting of its axis relative to the axis of said reception housing, said nut hence being of “poly-axial” type. Moreover, at least one of the head screws then comprise a head prolongated by a body fitted—with a thread for fastening in the bone, and—with a thread intended for co-operating with the thread of the related plate nut.

Within the framework of this advantageous embodiment:
the first drilling guide is fitted with a threaded insert adapted for being screwed in one of the nuts of the plate head, and
said second drilling guide is adapted for co-operating with one of the orifices of said targeting guide, abutting against one of said nuts of said plate head.

The first drilling guide comprises advantageously a head part shaped and sized to come through the through-orifices of the targeting guide, with a clearance allowing it a degree of freedom according a tilting range, to permit the implementation of the poly-axial technique of the associated head screws, through said orifices of the targeting guide arranged onto the osteosynthesis plate.

Preferably still, the targeting guide is fitted with a single through-orifice for the fastening thereof on the osteosynthesis plate and it also comprises, on its bottom face intended for contacting the plate head, at least one toe or one recess intended for co-operating with one complementary toe or recess to match the top face of said plate head, to ensure correct centring of said targeting guide on said plate head.

According still to another particularity, the device offered to the surgeon comprises a set of positioning spindles or pegs; moreover, the targeting guide and the osteosynthesis plate (at the head part thereof), comprise matching orifices for letting through these positioning pegs.

The second drilling guide (used in combination with the targeting guide) comprises advantageously a graduation system intended for co-operating with a mark provided on the related drill bit, so as to determine the drilling depth performed, and thus to determine the suitable screw length to be used.

On the other hand, the device available to the surgeon comprises further advantageously a gauge capable of determining the length of the through drill-hole provided through the drilled bone, which gauge comprises a hollow cylindrical body whereof the insert is adapted for resting on the osteosynthesis plate, optionally through one of the orifices of the targeting guide, associated with a ram whereof one of the ends is fitted with a graduated system and whereof the other end is fitted with a positioning hook.

The invention will be illustrated further, without being limited thereto, by the following description of different operating techniques, using the set of devices according to the invention, especially in relation with the appended drawings wherein:

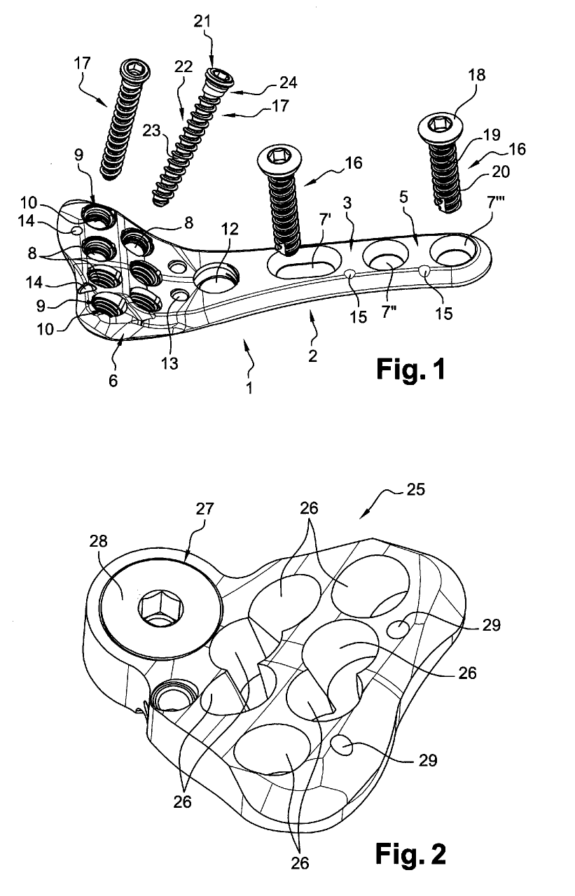

FIG. 1 is a perspective view of one osteosynthesis plate part of the device offered;

FIG. 2 is a perspective view of the targeting guide part of the device offered;

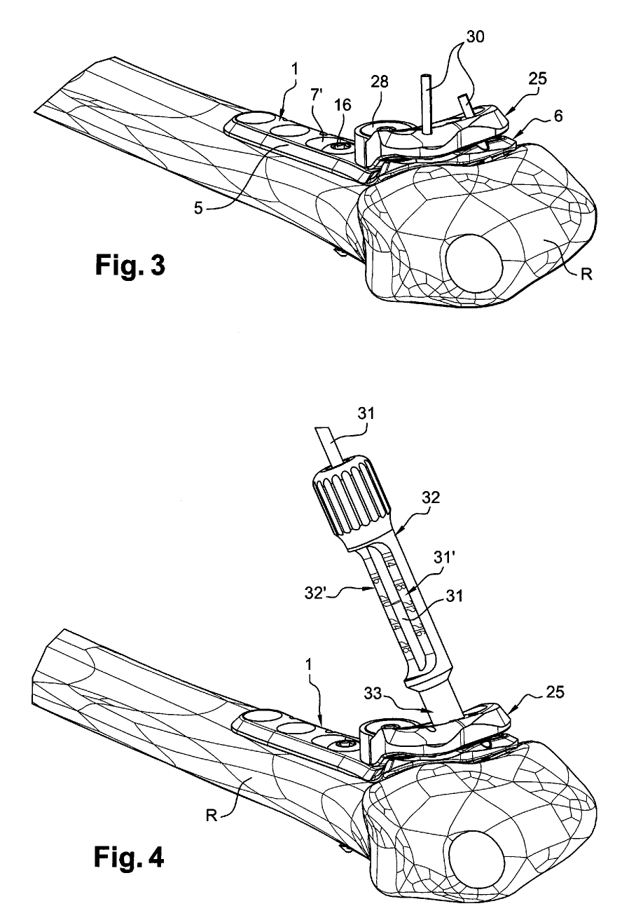

FIG. 3 the plate/targeting guide assembly during correct positioning on the end of a radius, in combination with positioning pegs;

FIG. 4 illustrates the drilling of a drill-hole in the end of the radius, for positioning a head screw according to a mono-axial technique using the targeting guide and a suitable drilling guide;

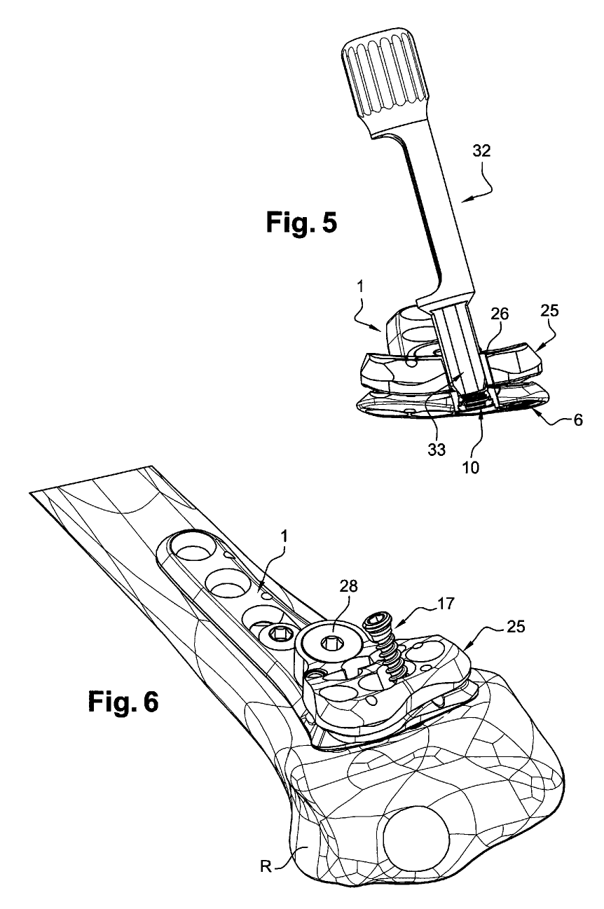

FIG. 5 is a sectional view based on FIG. 4, whereof the sectional plan is arranged through the targeting guide orifice in which is arranged the drilling guide;

FIG. 6 shows the placement of a head screw through the targeting guide, after drilling the drill-hole illustrated on FIGS. 4 and 5;

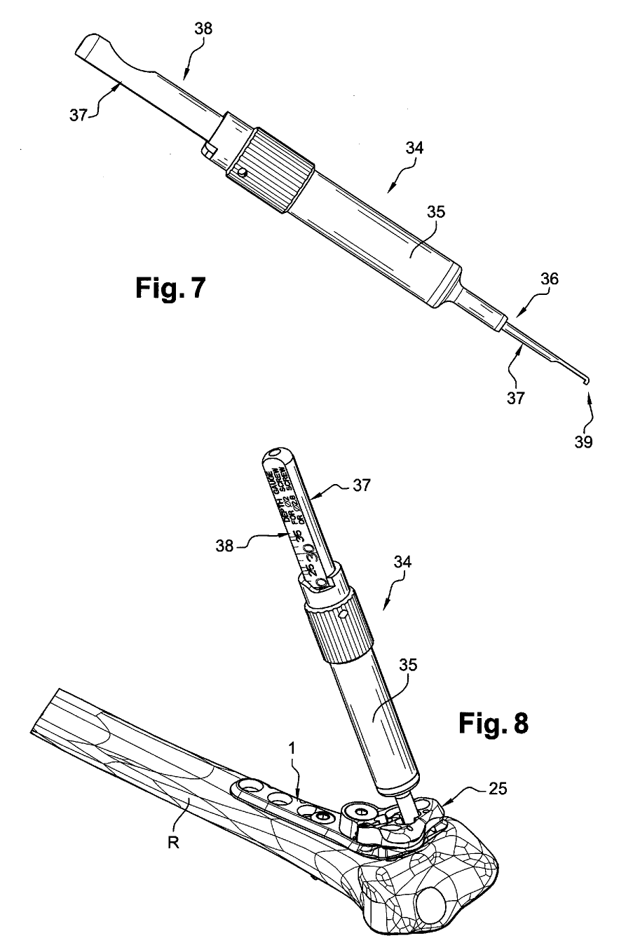

FIG. 7 shows the gauge enabling to determine the length of a through drill-hole provided in the bone;

FIG. 8 shows the use of the gauge illustrated on FIG. 7, on the plate/targeting guide assembly;

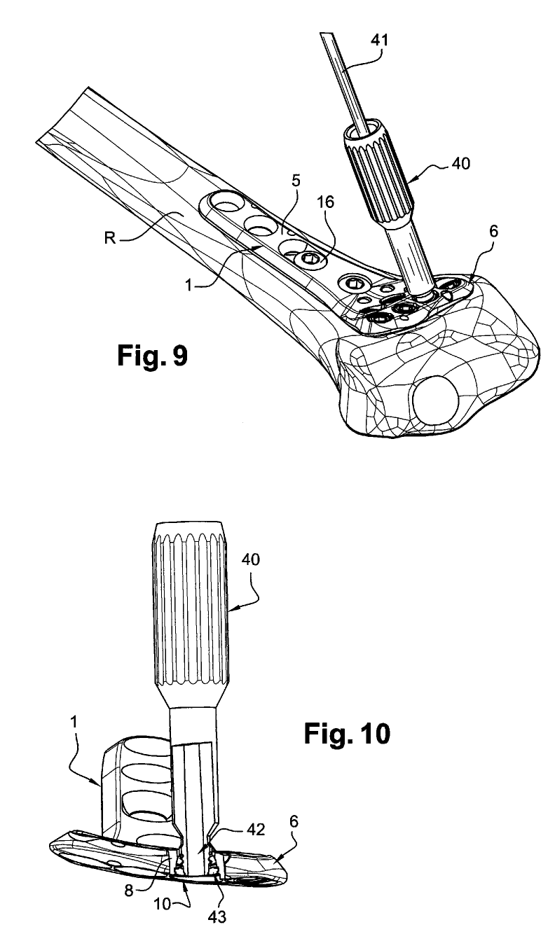

FIG. 9 shows the use of the other drilling guide for the implementation of the poly-axial fastening technique;

FIG. 10 is a sectional view of FIG. 9, whereof the sectional plan is arranged at the osteosynthesis plate orifice in which is positioned the drilling guide;

FIG. 11 is a view of the osteosynthesis plate mounted correctly on the radius with the set of its fastening screws;

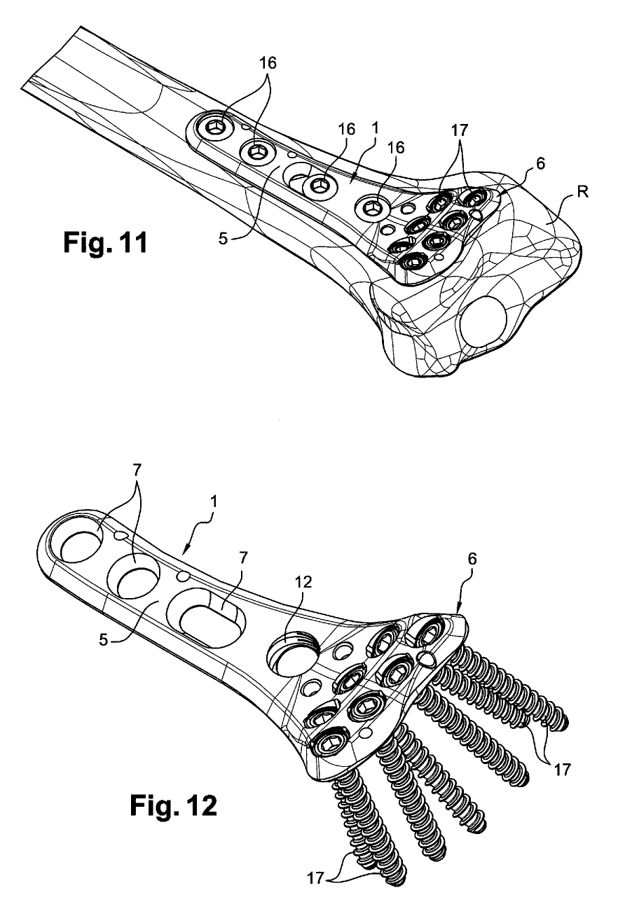

FIG. 12 is an individual view of the plate, showing the implantation of the different head screws;

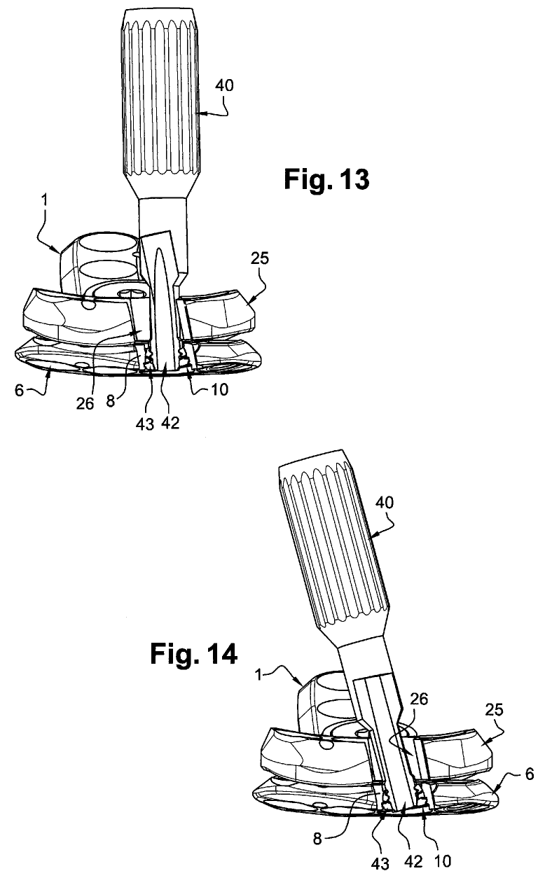

FIG. 13 is a sectional view illustrating a variant embodiment in which the drilling guide for implementing the poly-axial technique can be used with the targeting guide arranged onto the osteosynthesis plate;

FIG. 14 is a sectional view according to FIG. 13, which shows another tilting of the drilling guide, illustrating the poly-axial character of the technique, even when the targeting guide is present;

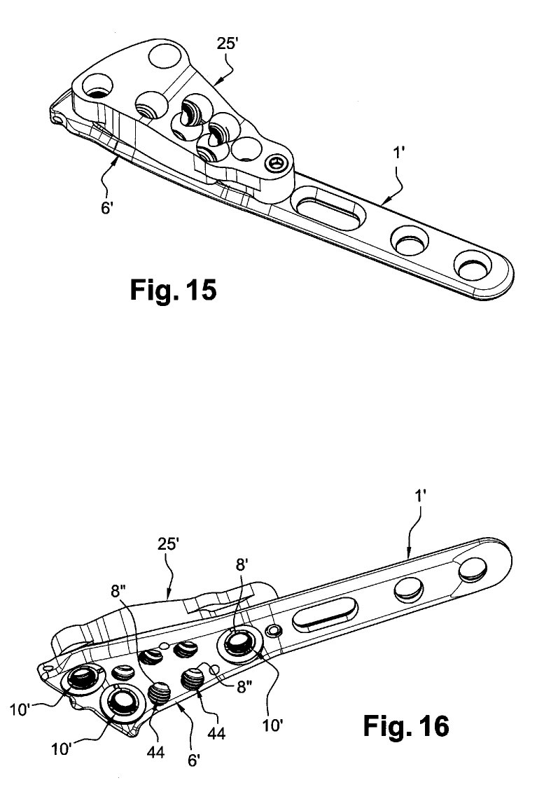

FIGS. 15 and 16 are perspective views, respectively from above and bellow, of an osteosynthesis plate variant eventually part of the material proposed, associated with the targeting guide.

As illustrated on the FIGS. 1 to 12, the device according to the invention offered to the surgeon, for the reduction of a distal radius fracture, comprises in particular an osteosynthesis plate, a set of diaphysial screws, a set of epiphysial screws, a targeting guide, two drilling guides, a set of positioning pegs and a drilling device (surgical motor and a set of suitable drill bits).

The osteosynthesis plate 1 illustrated individually on FIG. 1, comprises a bottom face 2 intended for contacting the bone material and a top face 3. This plate 1 is formed of an elongated body part 5 suitable for being placed on the diaphysis of the bone, and of a one-piece head part 6, suitable for being placed on the bone epiphysis.

The body part 5 comprises a plurality of aligned through-orifices 7′, 7″, 7″′ (three in this case), whereof one 7′ exhibits an oblong elongated shape. The head part 6 comprises for its own part a plurality of through-orifices 8 provided on two lines substantially perpendicular to the axis of the body part 5. The (end) distal line is composed of an alignment of four orifices 8 whereas the proximal line is composed of an alignment of three orifices 8.

Each head orifice 8 comprises a housing 9 for receiving a “poly-axial” nut 10, for instance as described in Documents FR-A-2 832 308 and FR-07 02777.

This nut 10 comprises a threaded central orifice and a spherical contact surface (not visible on FIG. 1) suitable for co-operating with a complementary spherical surface provided in the reception housing 9 (also not visible on FIG. 1) for conferring its poly-axial character thereto (i.e. for conferring a degree of tilting freedom thereto) along an admissible predetermined tilting range. Tenon/mortise type means are also provided to lock the nuts 10 into rotation in their reception housing 9 (these means do not appear on FIG. 1).

At the linking zone between the body part 5 and the head part 6, the plate 1 comprises a complementary orifice 12 fitted with a thread.

Moreover, on the top face of the head part 6, just in front of the complementary orifice 12, the presence of two small through-mortises 13 in the form of cylindrical reservations can be noted.

The distal end of the head part 6 comprises further two small unthreaded through-orifices 14. On one of the sides of the body part 5, the presence of two small unthreaded through-orifices 15 can also be noted, intended for receiving temporarily and, if required, pegs which may hold the plate on the diaphysis before the placement of the fastening screw.

The device according to the invention also comprises a set of screws 16 for attaching on the bone the plate body 5 via through-orifices 7 (so-called “body screws”), and a set of screws 17 for attaching on the bone the plate head 6 via through-orifices 8 (so-called “head screws”). The body screws 16 are each formed of a head 18 prolongated by a body 19 fitted with a bone thread 20. The head screws 17 are each formed of a head 21 prolongated by a body 22 fitted with a bone thread 23 and a thread 24 intended for co-operating with the thread of the orifice of one of the nuts 10.

On the other hand, the set of devices according to the invention also comprises a known targeting guide device 25, as illustrated on FIG. 2. This targeting guide 25 consists of a block of material having for instance a thickness of the order of 4 to 6 mm, whereof the general shape corresponds approximately to that of the head part 6 of the osteosynthesis plate 1, and which is fitted with through-orifices 26. The number of these through-orifices 26 is identical to that of the orifices 8 of the head part 6 of the plate 1 (i.e., seven) and these orifices 26 are provided each for matching one of these head orifices 8, when said targeting guide 25 is mounted on said plate 1.

The targeting guide 25 comprises further an orifice 27 intended for being positioned opposite the threaded complementary orifice 12 of the plate 1, for the positioning of a screw 28 enabling the removable fixture thereof on said plate 1. The centred correct positioning of the targeting guide 25 on the plate head 6 is obtained by means of toes or of cylindrical single-piece protruding studs (not visible on FIG. 2) located on its bottom face and which are intended for penetrating the complementary reservations 13 aforementioned of the plate head 6.

The through-orifices 26 of the targeting guide 25 are cylindrical and unthreaded; they are each intended for acting as a guide for accurate orientation positioning of the head screws 17.

The axis of each of the orifices 26 is particular, oriented along a predetermined direction corresponding to the accurate direction optimum for the head screws 17 intended for being located in the matching head orifice 8, notably for current fractures, encountered relatively frequently.

The targeting guide 25 comprises further two small through-orifices 29 intended for matching the end orifices 14 of the plate head 6, as will be seen below, adapted for letting through positioning pegs.

For the reduction of a distal radius fracture of average complexity, the surgeon puts the targeting guide 25 on the plate head 6 by means of the fastening screw 28; the associated tenon/reservation assemblies aforementioned provide for centring and correct positioning of both elements relative to one another.

Then, he places as well as possible the osteosynthesis plate 1 on the radius R and he attaches the body part 5 on the bone structure by means of a body screw 16 in the oblong orifice 7′ (FIG. 3).

The surgeon then checks the plate for correct location by inserting two positioning pegs 30 in the couples of orifices 14-29 of the targeting guide 25 and of the plate head 6, as well as through the bone R, so as to make sure that they do not emerge into the joint of the radius. It is there a safety enabling to prevent the head screws 17 laid at a later stage from emerging into the joint of the radius.

The corresponding control on the positioning pegs 30 is provided by radiology or brightness amplifier.

If required, the positioning of the plate 1 is modified longitudinally by loosening the body screws 16 slightly before proceeding to a new check.

Once the plate 1 considered as located correctly, the body screw 16 is clamped.

The surgeon then has the possibility of placing at least some of the head screws 17 according to a “mono-axial” technique via the targeting guide 25.

For each of the corresponding screws 17, as illustrated on FIGS. 4 and 5, a drill-hole in the bone is drilled by means of a surgical motor (not represented) fitted with a drill bit 31, in combination with a drilling guide 32. The cylindrical end 33 of the drilling guide 32 is guided by one of the orifices 26 of the targeting guide 25 and it is arranged to abut against the matching nut 10 housed in the plate head 6. The cylindrical head 33 of the drilling guide 32 has a diameter corresponding, within the clearance, to the diameter of the orifices 26 of the targeting guide 25.

The matching drill-hole is provided until the head of the drill bit 31 reaches the opposite cortical of the bone (for correct hooking of the screw 17 with the longest possible grip).

A mark 31′ on the drill bit 31, associated with a graduation 32′ provided on the drilling guide 32 enable to know accurately the length of the head screw 17 which should then be used.

After the removing of the drill bit 31 and of the drilling guide 32, the corresponding screw 17 is placed (FIG. 6) through the related orifice 26 of the targeting guide 25. Once fully inserted, this screw 17 is compressed on the plate 1 and locked by the presence of the related nut 10.

The operation is repeated for all the head screws 17 that the surgeon wishes to implant according to this “mono-axial” technique.

This implemented facilitates substantially the surgeon's work and vastly limit the operating time of the osteosynthesis.

If the surgeon runs completely through the radius R when making the drill-hole for the screws, the device available to him comprises a gauge 34, illustrated individually on FIG. 7, suitable for determining the dimension of the screws 17 to be used.

This gauge 34 comprises a body 35 generally cylindrical and hollow in shape, whereof the head 36 is arranged for running through the corresponding orifice 26 of the targeting guide 25 and for resting on the top face 3 of the osteosynthesis plate 1. This gauge 34 comprises further a ram 37, housed in the hollow body 35, whereof the upper portion 38 is graduated and whereof the lower end is formed of a rod fitted with an end hook 39. It should be understood that once the gauge body 35 is resting on the plate 1, the ram 37 may be operated so as to hook the opposite cortical of the bone, through the drill-hole provided, this hooking enabling to determine the exact depth of the drill-hole by means of the graduation 38, facing the upper end of the hollow body 35, to define the adequate screw length (i.e. a screw whereof the end will reach the opposite cortical of the bone). FIG. 8 illustrates the use of this gauge 34 on the plate 1 fitted with the targeting guide 25.

Once the surgeon has placed all the screws 17 he wished to install in “mono-axial” technique, he removes the targeting guide 25 (simply by loosening the screw 28). It may then place the remaining head screws 17 according to a “poly-axial” technique.

He makes to this aim drill-holes by using a drilling guide 40 (FIGS. 9 and 10) associated with a surgical motor (not represented) fitted with an adapted drill bit 41 (only visible on FIG. 9). The head 42 of the drilling guide 40 is fitted with a thread 43 and it is screwed in the nut 10 associated with the corresponding head orifice 8; the surgeon adjusts the angularity of the drilling guide 40 for making correctly the drill-hole along the requested direction; he makes the drill-hole up to the opposite cortical of the bone and he uses the gauge 34 aforementioned, illustrated on FIG. 7, to determine the suitable screw length to be used.

The surgeon may complete the placement of the head screws 17 while repeating this succession of operations.

The attachment of the osteosynthesis plate 1 is completed by placing the remaining body screws 16. It should be noted here that the complementary orifice 12 of the osteosynthesis plate 1 is used advantageously for fixing a body screw 16 identical to the others.

An osteosynthesis plate 1 laid and attached on the radius R as illustrated on FIG. 11 is obtained.

FIG. 12 illustrates a possible angular configuration of the different head screws 17.

According to the type of fracture at hand, the set of devices according to the invention may also be used by the surgeon for laying all the head screws 17 in mono-axial technique, by using solely the targeting guide 25 (in particular for simple fractures), or for laying the set of head screws 17 in poly-axial technique, by using solely the drilling guide 40 (in particular for very complex fractures).

When he wishes to use both techniques, the surgeon starts preferably with the mono-axial technique so as to use a targeting guide 25 pre-positioned on the osteosynthesis plate 1 and so as to use the poly-axial technique on an osteosynthesis plate 1 already secured in position on the bone.

In accordance with the above embodiment, the drilling guide 40 is used after the removing of the targeting guide 25.

According a variant embodiment illustrated FIGS. 13 and 14, le targeting guide 25 and the drilling guide 40 are structured to allow the implantation of the head screws 17 thanks to a poly-axial technique while maintaining the targeting guide 25 in position onto the osteosynthesis plate 1.

As illustrated FIGS. 13 and 14, the head 42 of the drilling guide 40 is shaped and sized to allow its passage through the orifices 26 of the targeting guide 25, and that while preserving some possible angulation range compare to the axis of the stacked orifices 8 and 26. This possible angulation range corresponds advantageously to the one allowed by the nuts 10 in their reception housings 9. The presence of the targeting guide 25 allows then the contours of the orifices 26 to constitute a mechanical stop preventing the surgeon to go beyond the admissible angulation range.

The FIGS. 13 and 14 show two different angulations of the drilling guide 40 to illustrate this principle.

The fact to allow the working thanks to a poly-axial technique, while preserving the targeting guide 25 in position onto the osteosynthesis plate, makes the surgeon intervention easier and faster. Indeed, he can use the targeting guide 25 to start working in mono-axial technique (easy and fast), then, during the operation, in function of the difficulties encountered or of the detected complexity at the fracture, changing without problems, very quickly, to a poly-axial implantation technique of one or several screws, without needing to remove said targeting guide 25.

After the implementation of the poly-axial technique for one or several screws, the surgeon can then come back, also easily, to a mono-axial implantation technique, the targeting guide being already in position.

The present invention can be useful for the reduction of epiphysis fractures, other than the ones of the distal radius, for instance for the proximal humerus, the distal femur, etc.

The structure of the osteosynthesis plate is suitable to the considered use. Its general form will be in particular function of the physical spatial configuration of the fracture zone intended to be reduced. Function of the case, not all the head orifices 8 can be equipped with receiving means of screws having a poly-axial character.

For instance, the FIGS. 15 and 16 show an epiphysis humeral plate 1′ equipped of its removable targeting guide 25′ and whereof the head 6′ is provided with orifices equipped, for some of them 8′, of nuts 10′ for the reception of screws having a poly-axial character, and for the others 8″, a simple inner thread 44 for the reception of screws having a mono-axial character.

1. A device for reducing a fracture, in particular a fracture at the bone epiphyses, characterised in that it comprises, in combination:
an osteosynthesis supporting plate (1, 1′) including a bottom face (2) and a top face (3), said bottom face (2) intended for being located against the receiving bone material (R), which plate (1, 1′) comprises an elongated body part (5), prolongated by a one-piece head part (6, 6′), said body part (5) including a plurality of through-orifices (7) and said head part (6, 6′) including a plurality of through-orifices (8; 8′, 8″), at least some of them enabling to accommodate poly-axial type screws, i.e. capable of being implanted along an admissible predetermined tilting range, said plate (1, 1′) still comprising, at the linking zone between the body part (5) and the head part (6, 6′), at least one complementary threaded orifice (12),
a set of screws (16) for fastening into the bone, intended for being inserted into said through-orifices (7) of the plate body (5), so-called “body screws”, for attaching said plate body (5) to the surface of the bone, which body screws (16) comprise a head (18) prolongated by a body (19) fitted with a thread (20) for fastening into the bone,
a set of screws (17) for fastening into the bone, intended for being inserted into said through-orifices (8; 8′, 8″) of the plate head (6, 6′), so-called “head screws”, for attaching said plate head (6) to the surface of the bone,
a targeting guide type device (25, 25′) intended for being positioned on the top face (3) of said plate head (6, 6′), to enable set tilting positioning of said head screws (17), which targeting guide (25, 25′) consists in a piece of matter comprising a plurality of through-orifices (26), identical in number to those (8; 8′, 8″) of said plate head (6, 6′), each intended for matching one of said orifices (8; 8′, 8″), which through-orifices (19) are adapted for acting as a guide for said head screws (17), and which targeting guide (25, 25′) still comprises at least one through-orifice (27) intended for being positioned in the extension of said complementary orifice (12) of the plate head (6, 6′), for its removable fixture thereon by means of an adapted fastening screw (28),
means for drilling orifices, of surgical motor type associated with a set of suitable drill bits (31, 41),
a first drilling guide (40), adapted for co-operating with the orifices (8; 8′, 8″) of said plate head (6, 6′), to enable drilling into the bone a drill-hole for receiving said head screws (17), according a poly-axial technique,
a second drilling guide (32), adapted for co-operating with the orifices (26) of said targeting guide (25, 25′) positioned on said plate head (6, 6′), to enable drilling into the bone a drill-hole for receiving said head screws (17), according a mono-axial technique.

2. A device according to claim 1, characterised in that it comprises an osteosynthesis plate (1) whereof at least one the through-orifices (8; 8′, 8″) of the head part (6, 6′) is fitted with means for locking the associated head screw (17) on said plate (1, 1′), upon completion of the screwing operation into the receiving bone material.

3. A device according to claim 2, characterised in that at least one of the through-orifices (8; 8′, 8″) of the head part (6, 6′) of the osteosynthesis plate (1, 1′) comprises a housing (9) provided for accommodating and holding a nut (10), said nut (10, 10′) being blocked into rotation in its housing (9) relative to said supporting plate (1, 1′) and said nut (10, 10′) comprising a spherical contact surface capable of co-operating with a complementary spherical contact surface provided in its reception housing (9), to confer said nut (10, 10′) a degree of freedom in said housing (9), along an admissible predetermined tilting range, to enable tilting of its axis relative to the axis of said reception housing (9), said nut (10, 10′) hence being of “poly-axial” type, and in that at least one of these head screws (17) comprise a head (21) prolongated by a body (22) fitted—with a thread (23) for fastening into the bone, and—with a thread (24) intended for co-operating with the thread of the related plate nut (10, 10′).

4. A device according to claim 3, characterised in that:
said first drilling guide (40) is fitted with a threaded insert (42, 43) adapted for being screwed in one of the nuts (10, 10′) of the plate head (6, 6′), and
said second drilling guide (32) is adapted for co-operating with one of the orifices (26) of said targeting guide (25, 25′), abutting against one of said nuts (10) of said plate head (6, 6′).

5. A device according to any of the claims 1 to 4, characterised in that the first drilling guide (40) comprises a head part (42) shaped and sized to come through the through-orifices (26) of the targeting guide (25, 25′), that with a clearance allowing it a degree of freedom according a tilting range, to permit the implementation of the poly-axial technique of the associated head screw through said orifice (26) of the targeting guide (25, 25′) arranged onto the osteosynthesis plate (1, 1′).

6. A device according to any of the claims 1 to 5, characterised in that it comprises a targeting guide (25, 25′) fitted with a single through-orifice (27) for the attachment thereof on the osteosynthesis plate (1, 1′) and fitted, on its bottom face intended for contacting the plate head (6, 6′), with at least one toe or one recess intended for co-operating with one complementary toe or recess (13) provided to match on the top face (3) of said plate head (6, 6′).

7. A device according to any of the claims 1 to 6, characterised in that it comprises a set of positioning pegs (30), and in that the targeting guide (25, 25′) and the plate head (6, 6′) comprise matching orifices (14, 29) for letting through said positioning pegs (30).

8. A device according to any of the claims 1 to 7, characterised in that the second drilling guide (32) comprises a graduation system (32′) intended for co-operating with a mark (31′) provided on the related drill bit (31), so as to determine the drilling depth performed, and thus to determine the suitable screw length (17) to be used.

9. A device according to any of the claims 1 to 8, characterised in that it comprises a gauge (34) capable of determining the length of the through drill-hole provided through the drilled bone, which gauge (34) comprises a hollow cylindrical body (35) whereof the insert (36) is adapted for resting on the osteosynthesis plate (1, 1′), optionally through one of the orifices (26) of the targeting guide (25, 25′), associated with a ram (37) whereof one of the ends is fitted with a graduated system (38) and whereof the other end is fitted with a positioning hook (39).
